(19) United States
(12) Patent Application Publication
(10) Pub. No.: US 2021/0062175 A1
(43) Pub. Date: Mar. 4, 2021

(54) FIBRINOLYTIC ENZYME FROM AGROCYBE AEGERITA AND A PREPARATION METHOD THEREOF

(71) Applicant: QIQIHAR UNIVERSITY, QIQIHAR (CN)

(72) Inventors: XIAOLAN LIU, QIQIHAR (CN); GUANLONG LI, QIQIHAR (CN); XIQUN ZHENG, QIQIHAR (CN); YONGPING DENG, QIQIHAR (CN)

(21) Appl. No.: 16/990,897

(22) Filed: Aug. 11, 2020

(30) Foreign Application Priority Data
Sep. 2, 2019 (CN) 201910822101.X

(51) Int. Cl. C12N 9/58

(52) U.S. Cl. CPC C12N 9/58; C12Y 304/24072

(57) ABSTRACT

A fibrinolytic enzyme from Agrocybe aegerita and a preparation method thereof. The fibrinolytic enzyme from Agrocybe aegerita is composed of two subunits A and B, the molecular weight of the subunit A is 31.4 kDa; the molecular weight of the subunit B is 21.2 kDa; the twelve amino acids sequence of the N-terminal of the subunit A is shown in SEQ ID No. 1; the twelve amino acids sequence of the N-terminal of the subunit B is shown in SEQ ID No. 2. The optimal temperature for the fibrinolytic enzyme from Agrocybe aegerita provided in the invention is 47° C., which can maintain good activity at physiological pH of human. Fe²⁺ at different concentrations shows obvious inhibiting effects on the fibrinolytic enzyme from Agrocybe aegerita. The fibrinolytic enzyme from Agrocybe aegerita exhibits good thrombolytic properties, thus providing a research basis for the preparation of thrombolytic drugs and/or functional foods.

### CROSS REFERENCE TO RELATED APPLICATION

This application claims the priority of Chinese Patent Application No. 201910822101.X entitled “A FIBRINOLYTIC ENZYME FROM AGROCYBE AEGERITA AND A PREPARATION METHOD THEREOF”, filed with the Chinese Patent Office on Sep. 2, 2019, which is incorporated herein by reference in its entirety.

### TECHNICAL FIELD

The disclosure relates to the technological field of biological engineering, and specifically relates to a fibrinolytic enzyme from Agrocybe aegerita and a preparation method thereof.

### BACKGROUND

Thrombotic disease has seriously threatened human life and health, and its disability rate and fatality rate are increasing year by year, which is one of the leading causes of human death today. Thrombolytic therapy is the main method to treat and prevent thrombotic diseases. At present, thrombolytic drugs applied in clinical treatment have a certain effect, but there are still many drawbacks and being expensive. Therefore, it is an urgent need for modern medicine to develop new thrombolytic drugs that are efficient, fast, being capable of preventing re-embolization and reducing side effects. It is an important way for the development of thrombolytics drugs from various natural biological resources.

Agrocybe aegerita is a fungus belonging to the Basidiomycota, Agaricomycetes, Agaricomycetidae, Agaricales, Strophariaceae, Agrocybe, also known as Agrocybe cylindracea, Sorthern Poplar Mushroom, Agrocybe chaxingu, etc. Agrocybe aegerita is rich in amino acids and trace elements required for human body, and contains polysaccharides with anticancer activity. Agrocybe aegerita tastes delicious, has the effects of nourishing Yin and tonifying Yang and beauty, has unique curative effects on kidney deficiency, edema, rheumatism, etc., has better therapeutic functions for anticancer, antioxidation, and lowering blood pressure and lipid, which is a traditional medicinal and edible fungus, has an important medical value and is an important resource to be developed urgently.

For recently more than ten years, the applicant's team has been devoted to the preparation, purification and functional studies of fibrinolytic enzymes from microorganism, who ground breaking has purified four new fibrinolytic enzymes from Cordyceps militaris and Neurospora sitophila, which have been authorized the relevant inventive patent rights respectively, the Authorization Numbers are ZL201510159992.7, ZL201010204473.5, ZL200910072630.9, ZL200810137564.4, respectively. The four fibrinolytic enzymes are made by the fungal fermentation, which produces more metabolites, causing many steps for the subsequent purification of the fibrinolytic enzyme. Too many steps of purification will reduce the activity of the fibrinolytic enzyme, resulting in decreased fibrinolytic function. For recently years, the applicants have been trying to find a new source of the fibrinolytic enzyme with uncomplicated ingredients, which is easy to purify. The applicant's team found by screening that several edible fungus contain the fibrinolytic enzymes, the content of the fibrinolytic enzyme in Agrocybe aegerita is higher, up to 2780.05±34.32 U/g (in dry weight). It is worth nothing that the fibrinolytic enzyme from Agrocybe aegerita has not been reported at home and abroad.

### SUMMARY

In view of this, the object of the disclosure is to provide a fibrinolytic enzyme from Agrocybe aegerita and a preparation method thereof. The optimal temperature for the fibrinolytic enzyme from Agrocybe aegerita provided in the invention is 47° C., which can maintain good activity at the physiological pH of human, not only having good thrombolytic effects, but also being safe in use and with low cost on production, thus having a prospect of development and application.

For realizing the above purpose of the invention, the invention provides the following technical solution:

The invention provides a fibrinolytic enzyme from Agrocybe aegerita. The fibrinolytic enzyme from Agrocybe aegerita is composed of two subunits A and B, the molecular weight of the subunit A is 31.4 kDa; the molecular weight of the subunit B is 21.2 kDa; the twelve amino acids sequence of the N-terminal of the subunit A is shown in SEQ ID No. 1; the twelve amino acids sequence of the N-terminal of the subunit B is shown in SEQ ID No. 2.

The invention further provides a preparation method of the fibrinolytic enzyme from Agrocybe aegerita described in the above technical solution, including: extracting Agrocybe aegerita fruiting bodies with normal saline, to obtain an extract; performing a liquid-solid separation on the extract, the fibrinolytic enzyme in the resulting crude enzyme solution is separated successively through salting precipitation, gel chromatography, weak cation exchange chromatography, hydrophobic interaction chromatography and strong cation exchange chromatography.

Preferably, the ratio between the mass of Agrocybe aegerita fruiting bodies and the volume of normal saline is 1 kg:(8 to 12) L.

Preferably, the conditions for extraction include: the extraction time is S to 7 hours, and the extraction temperature is 3 to 5° C.

Preferably, the conditions for separation over the gel chromatographic column include: the eluent is a PBS buffer at 0.01 to 0.03 mol/L, the flow rate is 4 to 6 mL/min; and the pH of the eluent is 6.0.

Preferably, the separation by weak cation exchange chromatography is performed by linear elution, the starting buffer is the PBS buffer at 0.01 to 0.03 mol/L, the eluent is the PBS buffer at 0.01 to 0.03 mol/L containing 0 to 1 mol/L of NaCl; the pH of the PBS buffer is independently 6.0; and the flow rate is independently 1 to 3 mL/min.

Preferably, the separation by hydrophobic interaction chromatography is performed by linear elution, the starting buffer is the PBS buffer at 0.01 to 0.03 mol/containing ammonium sulfate at a saturation of 10%, the eluent is the PBS buffer at 0.01 to 0.03 mol/L containing ammonium sulfate at a saturation of 10% to 0%; and the pH of the PBS buffer is independently 7.4.

Preferably, the separation by strong cation exchange chromatography is performed by linear elution, the starting buffer is the PBS buffer at 0.01 to 0.03 mol/L, the eluent is the PBS buffer at 0.01 to 0.03 mol/L containing 0 to 1 mol/L of NaCl; the pH of the PBS buffer is independently 5.5.

The invention provides a fibrinolytic enzyme from Agrocybe aegerita and a preparation method thereof. The optimal temperature for the fibrinolytic enzyme from Agrocybe aegerita provided in the invention is 47° C., which can maintain good activity at physiological pH of human; Fe³⁺, K⁺ and Zn²⁺ all have obvious protective effects on this enzyme, while Fe²⁺ at different concentrations shows obvious inhibiting effects on this enzyme; the fibrinolytic enzyme from Agrocybe aegerita can not only degrade fibrin directly, but also activate plasminogen to degrade fibrin indirectly having good thrombolytic effects. The preparation method of the invention is simple, and low in cost.

The fibrinolytic enzyme from Agrocybe aegerita provided in the invention work well at the physiological pH of human body. It is indicated in the tests of Embodiments that the fibrinolytic enzyme from Agrocybe aegerita provided in the invention has good thrombolytic properties, which is capable of thrombolysis independently, also being capable of activating plasminogen for indirect thrombolysis, without obviously acute toxicity, thus having a prospect of development and application, and also adding a new member to the sparse fibrinolytic enzyme family. This invention provides a certain research basis for the application of the fibrinolytic enzyme from Agrocybe aegerita in the preparation of thrombolytic drugs and/or functional foods, also provides a new way for the comprehensive utilization of Agrocybe aegerita.

### BRIEF DESCRIPTION OF THE DRAWINGS

FIG. 1 is a diagram showing the dissolution of fibrin with the fibrinolytic enzyme from Agrocybe aegerita in Embodiment 1;

FIG. 2 is a diagram showing SDS-PAGE analysis of reduced human fibrinogen after digestion by the fibrinolytic enzyme from Agrocybe aegerita in Embodiment 1.

### DESCRIPTION OF THE EMBODIMENTS

The invention provides a fibrinolytic enzyme from Agrocybe aegerita, which is composed of two subunits A and B, the molecular weight of the subunit A is 31.4 kDa; the molecular weight of the subunit B is 21.2 kDa; the twelve amino acids sequence of the N-terminal of the subunit A is shown in SEQ ID No. 1; the twelve amino acids sequence of the N-terminal of the subunit B is shown in SEQ ID No. 2.

In the invention, the relative molecular mass of the fibrinolytic enzyme from Agrocybe aegerita is around 52 kDa.

In the invention, the sequence of SEQ ID No. 1 is shown as below: S-N-A-D-G-N-G-H-G-T-H-V. In the invention, the sequence of SEQ ID No. 2 is shown as below: A-I-V-T-Q-T-N-A-P-W-G-L.

The invention further provides a preparation method of the fibrinolytic enzyme from Agrocybe aegerita described in the above technical solution, including: extracting Agrocybe aegerita fruiting bodies with normal saline, to obtain an extract; performing a liquid-solid separation on the extract, the fibrinolytic enzyme in the resulting crude enzyme solution is separated successively through salting precipitation, gel chromatography, weak cation exchange chromatography, hydrophobic interaction chromatography and strong cation exchange chromatography.

In the invention, the Agrocybe aegerita fruiting bodies powders are obtained preferably by drying and then crushing the Agrocybe aegerita fruiting bodies. There is no special limitation on the crushing method in the invention, any conventional crushing method can be used. There is no special limitation on the source of the Agrocybe aegerita fruiting bodies, any conventional products on the market can be used. In the Embodiments of the invention, the Agrocybe aegerita fruiting bodies are preferably purchased from Gutian County Tianxian Agricultural Products Co., Ltd., the address of which is No. 75, Fenglin Road, Shuangzhu Village, Fengdu Town, Gutian County, and the bar code of the product is 6951370108152.

In the invention, the ratio between the mass of the Agrocybe aegerita fruiting bodies powders and the volume of normal saline is preferably 1 kg:(8 to 12) L, more preferably 1 kg:10 L. In the invention, the extraction time is preferably 5 to 7 hours, more preferably 6 hours. In the invention, the extraction temperature is preferably 3° C. to 5° C., more preferably 4° C.

There is no special limitation on the liquid-solid separation method in the invention, any conventional liquid-solid separation method can be used. In embodiments of the invention, the liquid-solid separation is performed preferably by centrifugation, the rotating speed of centrifugation is preferably 8000 to 12000 r/min, more preferably 10000 r/min; and the time for centrifugation is preferably 10 to 20 mins, more preferably 15 mins.

In the invention, the supernatant is preferably separated by salting out with ammonium sulfate, the ambient temperature for salting precipitation is preferably 3° C. to 5° C., more preferably 4° C.; the time for salting out is preferably 10 to 14 hours, more preferably 12 hours. In the invention, the aim of salting precipitation is to remove impure proteins.

In the invention, after salting precipitation and preferably over a liquid-solid separation, the precipitate is dissolved in the PBS buffer, and then the supernatant is separated by gel chromatography. In the invention, the gel chromatographic column is preferably Sephadex 0-25 gel chromatographic column. In the invention, the conditions for separation over the gel chromatographic column preferably include: the eluent is a PBS buffer at 0.01 to 0.03 mol/L, the flow rate is preferably 4 to 6 mL/min, more preferably 6 mL/min; and the pH of the eluent is preferably 6.0. In the invention, the eluent is preferably the PBS buffer at 0.02 mol/L. In the invention, the aim of separation by gel chromatography is to decolor and remove excessive ammonium sulfate.

In the invention, the active components obtained from separation by gel chromatography is preferably further separated by weak cation exchange chromatography. In the invention, the weak cation exchange chromatographic column is preferably CM-Sepharose Fast Flow weak cation exchange chromatographic column. In the invention, the separation by weak cation exchange chromatographic column is preferably performed by linear elution, the starting buffer solution is preferably the PBS buffer at 0.01 to 0.03 mol/L, more preferably the PBS buffer at 0.02 mol/L; the eluent is the PBS buffer at 0.01 to 0.03 mol/L containing 0 to 1 mol/L of NaCl, more preferably the PBS buffer at 0.02 mol/L containing 0 to 0.8 mol/L of NaCl; the pH of the PBS buffer is preferably 6.0 independently; and the flow rate is preferably 1 to 3 mL/min independently, more preferably 2 mL/min.

In the invention, the active components obtained from the separation by weak cation exchange chromatography is preferably further separated by hydrophobic interaction chromatography. In the invention, the hydrophobic interaction chromatographic column is preferably Source 15PHE hydrophobic interaction chromatographic column. In the invention, the separation by hydrophobic interaction chromatographic column is preferably performed by linear elution, the starting buffer solution is preferably the PBS buffer at 0.01 to 0.03 mol/L, more preferably the PBS buffer at 0.02 mol/L; the eluent is the PBS buffer at 0.01 to 0.03 mol/L containing ammonium sulfate at the saturation of 10% to 0%, preferably the PBS buffer at 0.02 mol/L containing ammonium sulfate at the saturation of 10% to 0%; the pH of the PBS buffer is preferably 7.4 independently.

In the invention, the active components obtained from the separation by hydrophobic interaction chromatography is preferably further separated by strong cation exchange chromatography. In the invention, the strong cation exchange chromatographic column is preferably Mono S 5/50 strong cation exchange chromatographic column. In the invention, the separation by strong cation exchange chromatography is preferably performed by linear gradient elution, the starting buffer is preferably the PBS buffer at 0.01 to 0.03 mol/L, more preferably the PBS buffer at 0.02 mol/L; the eluent is the PBS buffer at 0.01 to 0.03 mol/L containing 04 to 0.9 mol/L of NaCl, preferably the PBS buffer at 0.02 mol/L containing 0 to 0.8 mol/L of NaCl; the pH of the PBS buffer is preferably 5.5 independently.

The technical scheme of the invention will be clearly and completely described below in combination with embodiments in the invention. It is obvious that the embodiments described are only some embodiments of the invention and not all the embodiments. Based on the embodiments in the invention, all the other embodiments obtained by persons with ordinary skills in the art without creative works are all in the protection scope of the invention.

### Embodiment 1

Preparation of the Fibrinolytic Enzyme from Agrocybe aegerita

1) Preparation of crude enzyme solution: Agrocybe aegerita fruiting bodies are dried and crushed, 0.9% normal saline at a proportion of 1:10 (w/v) is added into the powders of Agrocybe aegerita fruiting bodies, which is extracted for 6 hours at 4° C., and centrifuged at 4° C. and 10000 r/min for 15 mins, with the supernatant being taken ready for use;

2) Salting out of the fibrinolytic enzyme: after centrifugation, the supernatant is adjusted until the saturation of ammonium sulfate reaching 80%, salted precipitation at 4° C. overnight, centrifuged at 4° C. and 10000 r/min for 15 mins, discarding the supernatant; the precipitates are dissolved in 50 mL of PBS (pH 6.0) buffer at 0.02 mol/L, and stored at 4° C. ready for use;

3) Separation by gel chromatography for desalination and decoloration: after salting precipitation, the enzyme solution is separated by Sephadex G-25 gel chromatographic column, the eluent is the PBS (pH 6.0) buffer at 0.02 mol/L, and the flow rate is 6 mL/min, collecting the active components;

4) Separation by weak cation exchange chromatography: the active components from the above step 3) are separated by CM-Sepharose Fast Flow weak cation exchange chromatographic column, the starting buffer is the PBS (pH 6.0) buffer at 0.02 mol/L, the eluent is the PBS (pH 6.0) buffer at 0.02 mol/L containing 0 to 0.8 mol/L of NaCl; then the linear elution is performed with increasing NaCl concentration, the flow rate is 2 mL/min, collecting the active components;

5) Separation by hydrophobic interaction chromatography: the active components from the above step 4) are separated by Source 15PHE hydrophobic interaction chromatography in a mode of linear elution, the starting buffer is the PBS (pH 7.4) buffer at 0.02 mol/L containing ammonium sulfate at the saturation of 10%, the eluent is the PBS (pH 7.4) buffer at 0.02 mol/L containing ammonium sulfate at the saturation of 10% to 0%. After loading, the linear elution is performed on the samples along the saturation of ammonium sulfate from 10% to 0 with a starting buffer of three times the volume of the column and the eluent of three times the volume of the column, collecting the active components;

6) Separation by strong cation exchange chromatography: the active components from the above step 5) are separated by Mono S 5/50 strong cation exchange chromatography, the starting buffer is the PBS (pH 5.5) buffer at 0.02 mol/L, and the eluent is the PBS (pH 5.5) buffer at 0.02 mol/L containing 0 to 0.8 mol/L of NaCl. After loading, the linear elution is performed on the samples with increasing NaCl concentration, the starting buffer of five times the volume of the column and the eluent of five times the volume of the column, collecting the active components to obtain the fibrinolytic enzyme from Agrocybe aegerita.

### Embodiment 2

Properties of the fibrinolytic enzyme from Agrocybe aegerita in Embodiment 1 after separation and purification are determined. The fibrin plate method is used to determine the related enzymatic properties.

The fibrinolytic enzyme from Agrocybe aegerita prepared in Embodiment 1 is composed of two subunits, the molecular weights of which are 31.4 kDa and 21.2 kDa, respectively; the optimal temperature is 47° C.; which can maintain good activity at physiological pH of human, being more stable in neutral and alkaline environment than in acid environment; Fe³⁺, K⁺ and Zn²⁺ all have obvious protective effects on this enzyme, while Fe²⁺ at different concentrations show obvious inhibiting effects on the fibrinolytic enzyme from Agrocybe aegerita. The fibrinolytic enzyme from Agrocybe aegerita can not only degrade fibrin directly, but also activate plasminogen to degrade fibrin indirectly, and can further degrade α, β, and γ chains of human fibrinogen in turn; the two sequences of twelve amino acids of the N-terminal of the two subunits are determined by the method of the Edman degradation as: \textcircled{1} S-N-A-D-G-N-G-H-G-T-H-V (SEQ ID No. 1), and \textcircled{2} A-I-V-T-Q-T-N-A-P-W-G-L (SEQ ID No. 2), respectively.

### Embodiment 3

Fibrinolytic Activity of the Fibrinolytic Enzyme from Agrocybe aegerita Prepared in Embodiment 1

A fibrin plate method is used to examine the dissolvability of this enzyme on fibrin.

A. Fibrin Plate Method

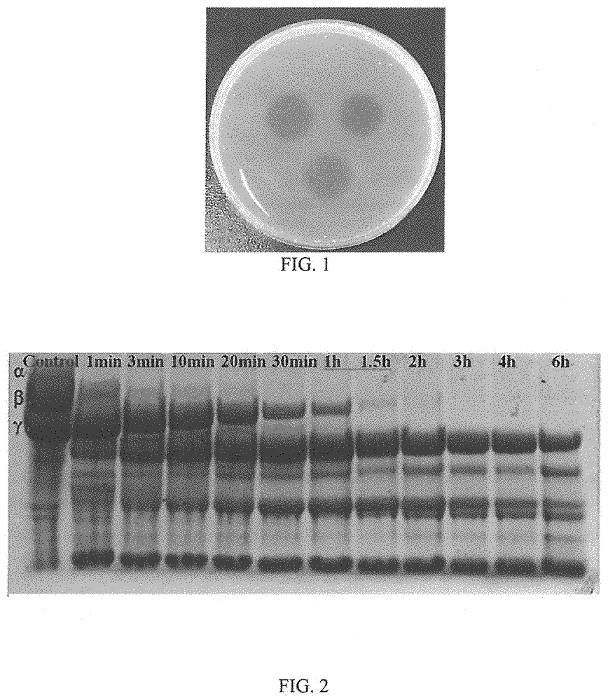

A fibrin plate contains fibrinogen (commercial fibrinogen may contain fibrin) and thrombin. Soluble fibrinogen forms fibrin monomer in the presence of thrombin, the fibrin monomer aggregates spontaneously to form visible fibrin gel. The fibrinolytic enzyme solution from Agrocybe aegerita is added onto the gel surface of this plate and kept at 37° C. cultured for a short time, then the fibrinolytic enzyme from Agrocybe aegerita would dissolve the fibrin, forming a transparent circle visible to the naked eyes on the gel surface of the plate, as shown in FIG. 1.

B. SDS-PAGE Electrophoresis

The fibrinolytic enzyme from Agrocybe aegerita prepared in Embodiment 1 is mixed evenly with human fibrinogen in equal volumes, and reacted in a water bath at 37° C. The degradation of human fibrinogen with the fibrinolytic enzyme from Agrocybe aegerita is detected by the SDS-PAGE analysis, with the degradation map shown in FIG. 2 (from left to right, the lanes represent, in order, human fibrinogen and 1 min, 3 min, 10 min, 20 min, 30 min, 1 h, 1.5 h, 2 h, 3 h, 4 h, 6 h after the fibrinolytic enzyme from Agrocybe aegerita mixing with fibrinogen). The α chain is degraded completely after 1 min, the β chain is degraded completely after 10 mins, and the γ chain is degraded completely after 2 hours, the degradation pattern of fibrinogen chain by the enzyme is identical to the plasmin.

It can be seen that the fibrinolytic enzyme from Agrocybe aegerita of the invention can dissolve fibrin and can degrade α, β and γ chains of human fibrinogen in order.

The foregoing descriptions are only the preferred embodiments of the invention. It should be noted that for persons with ordinary skills in the art, several improvements and modifications can be made to the invention without deviating from its principle, which are all considered as the protection scope of the invention.

1. A fibrinolytic enzyme from Agrocybe aegerita, wherein the fibrinolytic enzyme from Agrocybe aegerita is composed of two subunits A and B, the molecular weight of the subunit A is 31.4 kDa; the molecular weight of the subunit B is 21.2 kDa; the twelve amino acids sequence of the N-terminal of the subunit A is shown in SEQ ID No. 1; the twelve amino acids sequence of the N-terminal of the subunit B is shown in SEQ ID No. 2.

2. A preparation method for the fibrinolytic enzyme from Agrocybe aegerita of claim 1, comprising: extracting Agrocybe aegerita fruiting bodies with normal saline, to obtain an extract; performing a liquid-solid separation on the extract, the fibrinolytic enzyme in the resulting crude enzyme solution is separated successively through salting precipitation, gel chromatography, weak cation exchange chromatography, hydrophobic interaction chromatography and strong cation exchange chromatography.

3. The preparation method according to claim 2, wherein the ratio between the mass of Agrocybe aegerita fruiting bodies and the volume of normal saline is 1 kg:(8 to 12) L.

4. The preparation method according to claim 2, wherein the conditions for extraction comprise: the extraction time is 5 to 7 hours, and the extraction temperature is 3 to 5° C.

5. The preparation method according to claim 2, wherein the conditions for separation over the gel chromatographic column comprise: the eluent is a PBS buffer solution at 0.01 to 0.03 mol/L, the flow rate is 4 to 6 mL/min; and the pH of the PBS buffer is independently 6.0.

6. The preparation method according to claim 2, wherein the separation by weak cation exchange chromatography is performed by linear elution, the starting buffer is the PBS buffer at 0.01 to 0.03 mol/L, the eluent is the PBS buffer at 0.01 to 0.03 mol/L containing 04 to 1 mol/L of NaCl; the pH of the PBS buffer is independently 6.0; and the flow rate is independently 1 to 3 mL/min.

7. The preparation method according to claim 2, wherein the separation by hydrophobic interaction chromatography is performed by linear elution, the starting buffer is the PBS buffer at 0.01 to 0.03 mol/L containing ammonium sulfate at a saturation of 10%, the eluent is the PBS buffer at 0.01 to 0.03 mol/L containing ammonium sulfate at a saturation of 10% to 0%; and the pH of the PBS buffer is independently 7.4.

8. The preparation method according to claim 2, wherein the separation by strong cation exchange chromatography is performed by linear elution, the starting buffer is the PBS buffer at 0.01 to 0.03 mol/L, the eluent is the PBS buffer at 0.01 to 0.03 mol/L containing 04 to 1 mol/L of NaCl; and the pH of the PBS buffer Is independently 5.5.


SEQUENCE LISTING (2 sequences)

SEQ ID NO: 1 - protein, 12 aa, artificial sequence
  the twelve amino acids sequence of the N-terminal of the subunit A
SNADGNGHGTHV

SEQ ID NO: 2 - protein, 12 aa, artificial sequence
  the twelve amino acids sequence of the N-terminal of the subunit B
AIVTQTNAPWGL